(19) United States
(12) Patent Application Publication
(10) Pub. No.: US 2024/0091593 A1
(43) Pub. Date: Mar. 21, 2024

(54) System and Method for Strength Training

(71) Applicant: Christian Palmer, Branford, CT (US)

(72) Inventor: Christian Palmer, Branford, CT (US)

(21) Appl. No.: 18/274,984

(22) PCT Filed: Apr. 12, 2022
(86) PCT No.: PCT/US2022/024442

§ 371 Date: Jul. 28, 2023

Related U.S. Application Data
(60) Provisional application No. 63/174,068, filed on Apr. 13, 2021.

(51) Int. Cl. A63B 24/00; G16H 20/30

(52) U.S. Cl. CPC A63B 24/0075; G16H 20/30; A63B 2024/0068; A63B 2230/60

(57) ABSTRACT

Athletic strength training for both development and therapy involves compound athletic movements, like those specific to a sport such as throwing a pitch or those at the core of most athletic programs; like the popular powerlifting movements used for general strength training, back squat, bench press, and deadlift. By creation of reference sets from elite athletes ideal biomechanics profiles for these compound movements may be created. Thus establishing both a profile for assessing target athletes through a personal analysis as well as to prescribe training for target athletes. This reduces injury risks during development and athletic performance, accelerates strength development, and allows novel therapeutic approaches to injuries.

### TECHNICAL FIELD

The disclosure relates in general to a biomechanics analyzing system and a biomechanics analyzing method for athletic strength development, and more particularly to a system and a method for analyzing biomechanics through the use of calculating the relative weakest muscle in a group for prescription of a training regimen.

### BACKGROUND ART

In the fields of personal and athletic training compound movements are at the core of most athletic programs; like the popular powerlifting movements used for general strength training, back squat, bench press, and deadlift. These compound movements, like all compound exercise movements, are made from three types of muscles involved in the movement; the agonist or prime mover, the synergist or assisting muscles of the prime movers, and the stabilizing muscles of both the prime and assisting muscles. Each group both plays a unique role in all compound exercise, and can be targeted through isolation exercise. The difference between isolation and compound exercise here being the contraction of the muscles during strength exercise, where the latter have disproportionately few muscles show electrochemical activation as measured by mean amplitude in electromyography (EMG). Subjectively from the athletes experience the exercises are the ones which stress the muscle or muscles during exercise.

Muscular balance within these movements is essential to continued strength training with strength improvement, injury prevention, and injury rehabilitation. A common problem with strength training is continuing to improve without over training the relatively stronger muscles within the movement. Often, these exercises are limited by the weakest muscle or muscle group. As the muscles approach failure during exercise, over development of the relatively stronger muscles over time leads to an imbalance in biomechanics both during the movement and during general activity. Eventually this imbalance will progress to a risk factor for injury overtime leading to injuries during athletic training as the imbalance creates an irregular range of motion, and the greater relative load on the weaker muscles expresses in greater electrical activation at a quadratic relationship in measured EMG activation at a higher force level adding instability to the movement when the stronger muscles approach peak contraction. These imbalances express in a torn or strained muscle through over activation, a dropped weight as the stronger muscles fail without the proper support from muscles not trained to the same level in previous exercises, as well as during athletic performance from improper muscular development. An example is found in hamstring imbalance common in football players from reduced relative strength leading to injury as disclosed by Croisier, J. L., Ganteaume, S., Binet, J., Genty, M., & Ferret, J. M. (2008). Strength imbalances and prevention of hamstring injury in professional soccer players: a prospective study. The American journal of sports medicine, 36(8), 1469-1475. Further in the case of injury due to the lack of use muscular strength deteriorates, causing both a biomechanical imbalance and unpredictable biomechanics for targeting recovery exercise.

Currently the most popular method used for achieving muscular balance through strength development or rehabilitation are programs based on progressive overload training. Progressive overload is based on athletes exercising to a maximal weight for assessment, then exercising at a percentage of the maximal weight for reps of 3-5 with increasing increments each time they exercise. Another popular method used is structural balance assessments (SBA) as disclosed by Charles Poliquin in Poliquin, C. (1997). Poliquin principles: successful methods for strength and mass development, and Christian Thibedau in Nation, C. T., T. (2015, February 26). Know Your Ratios, Destroy Weaknesses. T NATION. https://www.t-nation.com/training/know-your-ratios-destroy-weaknesses/. SBA is a method of assessment of muscular balance by comparison of compound movements to reference lifts established through powerlifting records or experienced personal trainers from clients, to match recorded ratios of reference charts and clients in pursuit of the biomechanical ideal of reference athletes for clients.

The present invention is directed to a method which furthers previous methods of strength training and rehabilitation through a more comprehensive of assessment of muscular balance for use in prescription of exercises for development of strength. This is achieved through direct modeling of joint dynamics of any muscle group in general, sport or movement specific training, and injury rehabilitation with an assessment taking both target athlete specific segmental analysis into account as well as use of one rep max calculators.

### DISCLOSURE OF THE INVENTION

Typically athletes pursue a training regimen of progressive overload, or exercising to a maximal weight for assessment, then exercising at a percentage of the maximal weight for reps of 3-5 with increasing increments each time they exercise. This method does not account for other athletic activity or imbalances in biomechanics directly for injury prevention, or allow greater strength development through targeting the relatively weakest muscles through biomechanical ideals. Due to this athletes have injury risks during training or during athletic competition from improper development. Comprehensive assessments are possible through the costly isokinetic dynamometer, however both its cost and application to only torque of upper and lower limbs is limited in assessment. A previous strength training system which models muscular balance in a way accessible to a large number of athletes, SBA, for strength development and injury prevention has been limited to indirectly modeling joint dynamics from a small set of reference lifts to determine the biomechanical balance of the muscles involved in a movement. In the case of injury rehabilitation it is limited to prescribing weight at a very low percentage of weight used before injury for rehabilitation for an inability to model the strength of the weakest muscles. In general these assessments have been limited to prescription of exercises with the use of ratios between compound reference lifts and in some cases require inconvenient tempo based lifting to provide a full body routine, which does not apply to most athlete's regimens given the inability to target a specific muscular group's development and its incompatibility with the general strength development methods used in most athletic programs (back squat, bench press, and deadlift). Due to the limited number of reference lifts, they can not account for principles of kinesiology determined through segment analysis. Additionally they do not incorporate the use of one-rep max (1rm) calculators, which use a lower than maximal weight at repetition to failure to estimate one rep max, in determining a target athlete's strength level for a reference lift. At least one well known one rep max formula is the Epley formula where 1rm=weight lifted multiple by (1+ repetitions/30), this formula is disclosed with several other well known formulas by Richens, B., & Cleather, D. (2014). The relationship between the number of repetitions performed at given intensities is different in endurance and strength trained athletes Biology of Sport, 31(2), 157-161. Finally a SBA does not prescribe exercises to aid in rehabilitation of a specific muscle group as it is limited to broad assessments between compound exercises.

The limitations of SBA come across in the two most comprehensive forms of structural balance assessments used currently by athletic trainers in the list of compound movements from the disclosures above by Poliquin and Thibaudeau respectively. Neither list has the same number of exercises available or more than a few isolation exercises given their indirect modeling method, which does not allow flexibility based on athlete regimens outside of the provided list for general training, nor sport specific regimens, or injury rehabilitation prescriptions. Neither do they account for segment analysis which then means a general method of analysis is applied with less accuracy in reflecting a target athlete's biomechanics in a given assessment as the difference in athletes at a weight of 110 lbs and 310 lbs (e.g. arm limb weight differs on average in the bench press by 45 lbs from body weight 110 lbs to 310 lbs) has a very different ratio of biomechanical ideals based on the added limb weight to the given exercises.

The disclosure relates in general to a biomechanics analyzing system and a biomechanics analyzing method for strength training through individual exercises to model joint dynamics and muscular strength of a target individual compared to a biomechanical ideal. This allows a profile of the individual assessed to be created and compared to the ideal, allowing the prescription of an exercise regimen for the target individual. This assessment is created through the following formula for Predicted one rep max (P1rm): P1rm of a specific muscle is P1rm=(C/S)*(w), where (C) is the 1rm for a reference athlete or reference athletes for the complex exercise or where (C) is the 1rm or calculated 1rm for a reference athlete or reference athletes for the highest muscle assessed in isolation, (S) is the 1rm for a reference athlete or reference athletes for an isolation exercise associated with the compound exercise, therefore (C/S) is the biomechanical ideal defined by the ratio, and (w) is the calculated or measured 1rm of the isolation exercise. With this formula the muscles associated with a given compound exercise are exercised in isolation before being compared to establish a rank based on the P1rm associated with each individual exercise. The rank of the individual P1rm of exercises associated with the compound movement can be used for prescribing exercise, in order of priority from lowest to highest, to target the relatively weakest muscles in the exercise thereby preventing overdevelopment of the relatively stronger muscles. This assessment can be used for guidance for prescribing exercise over a set interval as recommended, or desired by the athlete (e.g. reassess every 6 weeks), given that the assessment is itself working the muscles to be developed during strength training.

According to the present disclosure the method provided allows use of individual exercises and compound exercises to determine muscular strength ideals in muscle groups through reference datasets of ideal biomechanical profiles, thus allowing for a biomechanics analysis in comparison. This comparison then is to be based on biomechanical ideal established through reference lifts. The reference lifts can be any complex multi muscle exercise which then allows for prescription of a regimen for development generally, as well as the way personal training programs or athletic programs for athletes in all sports train. The reference lifts determine the biomechanics profile through the P1rm formula.

Reference athletes or elite athletes are those which have reached peak or static strength in the related muscles of a compound movement as is typical with five years of development. In establishing a reference set for a compound movement through the P1rm formula there are two types of movements the method applies to, compound movements with a total weight lifted (including bodyweight) and compound movements with a force output that is without a max weight lifted (e.g. throwing a pitch). The ratio, (C/S) in the P1rm formula, represents the biomechanical ideal of the contributing muscles in isolation to the overall movement, thus an ideal biomechanical profile. Further in establishing a reference lift, physiology specific to reference athletes and target athletes may be used at increasing precision. As various factors affect the nature of force output in a given compound movement, for example limb weight differs in the bench press by 45 lbs from body weight 110 lbs to 310 lbs in men. A general list of considerations which may be controlled for are; sex, bodyweight, height, muscle mass, fat mass, visceral fat, fat free mass index, age, bone mass, skeletal muscle mass, maximum rate of oxygen consumption measured during incremental exercise or VO2 max, epigenetic clock, basal metabolic rate, glycemic index, and specific segment weight (limb, torso, etc. . . . ).

Another aspect of this disclosure is the use of a 1rm calculator which allows an athlete to use an individual or compound exercise with a lower weight for repetitions in assessing a reference lift, as is not an option in many implementations of SBA. This adds both to the safety through a full assessment of the strength of a muscle with a lesser weight allowing the athlete to maintain control of the weight, and allows an athlete to engage with the method of assessment with little prior experience.

### BRIEF DESCRIPTION OF DRAWINGS

FIG. 1 Discloses a process flow chart of the present invention according to the first embodiment of the invention.

FIG. 2 Discloses a process flow chart of the present invention according to the second embodiment of the invention.

FIG. 3 Discloses a process flow chart of the present invention according to the third embodiment of the invention.

FIG. 4 Discloses a process flow chart of the present invention according to the fourth embodiment of the invention.

FIG. 5 Discloses a process flow chart of the present invention according to the fifth embodiment of the invention.

FIG. 6 Discloses a process flow chart of the present invention according to the sixth embodiment of the invention.

### DESCRIPTION OF EMBODIMENTS

### First Embodiment

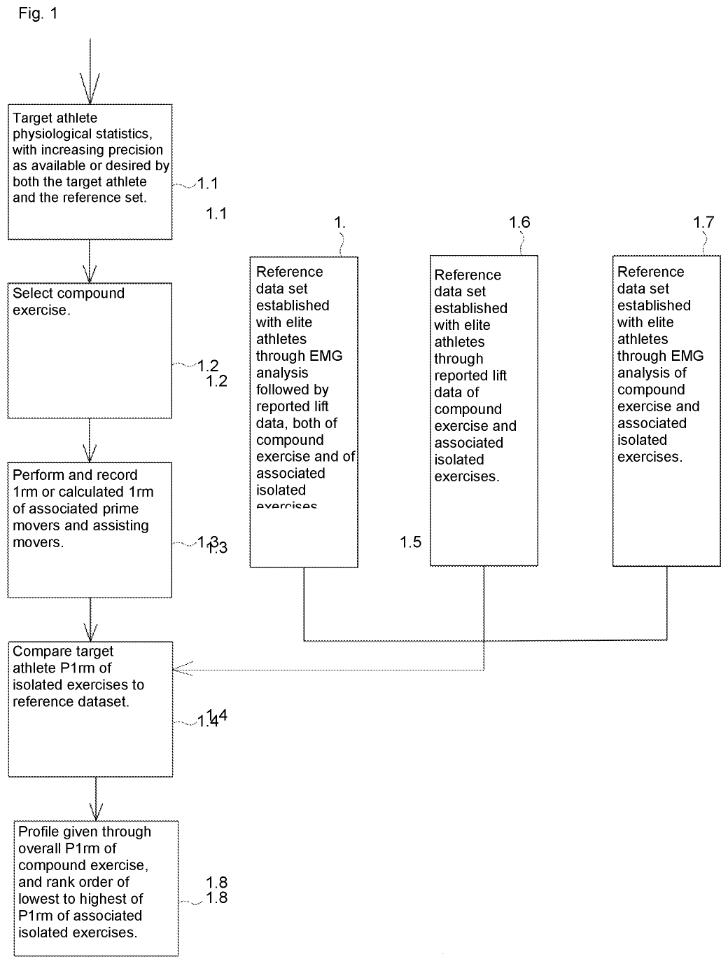

FIG. 1 is a process flowchart for the first embodiment of the invention; showing a method for a biomechanics analysis for strength training, by establishing both a reference muscular balance profile and further a target athlete profile for comparison, of a selected compound exercise with a total weight lifted. In the P1rm the weight ratio, not the actual strength of a muscle is the key factor, but rather the relative amount a muscular contraction produces force to lift a weight or perform an athletic movement. So while larger muscles such as the pectorals may be absolutely stronger than a smaller muscle such as the triceps in a movement like the bench press, the triceps may actually be stronger in a particular P1rm calculation as they may be used to greater degree with respect to the biomechanical ideal. Thus, when defining strong and weak muscles, the amount the muscle should be participating in the exercise is what is compared to the reference lift. Due to the nature this the assessment primarily uses exercises in isolation, so it is comprehensive in being able to account for individual physiological factors, and the exercises used during can be calculated through a 1rm formula. This analysis is done through providing a rank order and ideal balance ratio of each involved muscle from a reference dataset, as well as the lowest relative P1rm of any assessed muscle. A target athlete is then assessed for comparison.

In this embodiment one of three approaches to establishing reference lifts may be used (1.5, 1.6, 1.7—FIG. 1). In all approaches establishing a reference set for a compound movement through the P1rm formula there are two types of movements, compound movements with a total weight lifted (including bodyweight) and compound movements with a force output that is without a max weight lifted (e.g. throwing a pitch). In the first type of movement, as is used in this embodiment, (C) is represented by the 1rm or calculated 1rm of the actual weight lifted, and in the second (C) is defined as the highest weight in the reference lifts for all uses, as the overall P1rm is only a rank of the required lifts in determining the biomechanical ideal. For each reference set a desired level of precision could be selected based on the selected, although generally only sex and body weight are necessary, as these allow high precision through data aggregation. Further considerations that can be made are; height, muscle mass, fat mass, visceral fat, fat free mass index, age, bone mass, skeletal muscle mass, maximum rate of oxygen consumption measured during incremental exercise or VO2 max, epigenetic clock, basal metabolic rate, glycemic index, and specific segment weight (limb, torso, etc. . . . ).

The first approach to establishing a reference set for a compound movement through the P1rm formula is an EMG assessment of electrical activation, as determined by mean amplitude of the selected compound exercise. This determines the activated muscles during the movement. Then a, reference athlete or athletes, are assessed through working the involved muscles to failure through associated compound exercises in the first type of movement, and through isolated exercises for both types of movements. These exercises are recorded through the 1rm directly, or calculated through a formula (e.g epley). This establishes the strength for each muscle involved in the movement. From here a ratio, (C/S) in the P1rm formula, represents the biomechanical balance of the contributing muscles in isolation to the overall movement, thus a biomechanical ideal. By assessing a small set of reference athletes in the case of EMG assessment to establish an activation reference profile, followed by a larger number of reference athletes through 1rm and calculated 1rm based on the prior activation reference profile a database with high precision can be established for a biomechanics reference profile of a given movement.

A second approach, reported lift data, is only the use of reference athletes with a 1rm or calculated 1rm, for which a comparison of a reference athlete's respective total weight lifted and isolated exercises is made. This is limiting in the cases of movements that are not well established in known kinesiology of the muscles comprising the complex movement, unlike the well known core powerlifting movements (e.g. the deadlift's posterior chain muscles are well studied).

A third approach is through EMG activation. A reference athlete's respective compound and associated isolated movements are compared with any weight lifted for an associated isolation exercise. By recording the mean electrical amplitude of muscular contraction through electromyography with any weight, and then recording mean amplitude of peak contraction of the associated muscles of the compound exercise in isolation, a comparison of the two amplitudes and the weight in a ratio will predict the total weight that could be lifted by the muscle in isolation. This predicted weight can then be used to establish a reference data set through the P1rm formula.

In this embodiment the P1rm is calculated for each of the prime and assisting muscles involved in the selected compound exercise, while noting that this is a complete assessment as if these two muscle types are controlled for the stabilizing muscles will be as well with typical training methods. The lowest P1rm for the target athlete is determined from the weakest muscle in this group. Due to this, the lowest P1rm will be lower than an actual 1rm, as during any exercise other muscles are able to compensate for a weaker muscle in the exact way which causes development imbalances overtime as these stronger muscles over develop relatively. However, as the lowest P1rm is based on the weakest muscle, this allows the target athlete to complete a 1rm at a weight limit that is safe for all muscles involved both during the exercise, and over development as the weakest muscles are within a range of activation below their failure point during the compound exercise.

The first step in analysis is to have a target athlete's physiological assessment, with increasing precision as desired by and available by both, target athlete and the given reference set. After entering the chosen physiological statistics (1.1—FIG. 1), a matching reference set is chosen. For example if body weight and sex were entered, a reference set based on percentile weight used in a specific exercise at different weight classes and sex (e.g. by 10 lbs for sex), or at a specific bodyweight, is selected for a the balance profile of the selected compound exercise (1.2—FIG. 1).

The second step (1.3—FIG. 1) is recording the target athlete's P1rm for the compound movement through each muscle associated in isolation exercise of each prime and assisting muscle associated with the chosen compound exercise. P1rm is a novel prediction of a target athlete's 1rm for a given compound exercise, which is calculated without the target athlete actually completing the compound movement. The P1rm calculation can be based on the calculated 1rm, as determined by rep calculator formulas (e.g. Epley formula, Bryzycki formula, or estimation through heart rate), of the various muscles involved in the compound exercise assessed through isolation exercises which here targets the agonist or prime muscles, and synergist or assisting muscle. This isolation allows for a relative level of strength to be determined for each involved muscle with regards to the compound movement. These individual exercises that are completed at this step are recorded.

The third step is to take the previous isolated exercises recorded as the P1rm for the compound movement selected, which is done by comparing each individual muscle exercised in isolation to the reference lift dataset, giving an analysis by rank order (1.4—FIG. 1). For example, in a deadlift if a target recorded their rep count on a lying leg curl this would be inputted into the P1rm formula as (w) to calculate the P1rm by comparison to the ideal ratio at the selected weight represented by (C/S).

Finally, in the fourth step, an analysis is made (1.5—FIG. 1). First using the collective P1rm numbers established by comparing and then selecting the lowest P1rm from a given isolated muscle group of the selected compound movement, for a max weight in a personal training or athletic training regimen as representative of the relatively weakest muscle of the prime and assisting muscles, and therefore safe for use while maintaining proper biomechanics. Second the rank order, of lowest P1rm to highest P1rm, and the ratio of each muscle of the selected compound exercise measured in isolation will give a biomechanics profile.

While the first embodiment of the invention has been illustrated and described, it will be appreciated that implementation with other methods of health assessment or athletic training would not alter the spirit or scope of the invention. For example implementation into a larger health application or exercise program that uses muscular assessment as part of a larger program for either exercise or health tracking or prescription. Additionally one of ordinary skill will recognize that the step by step process of this embodiment is a calculation that can be readily implemented by a computer.

### Second Embodiment

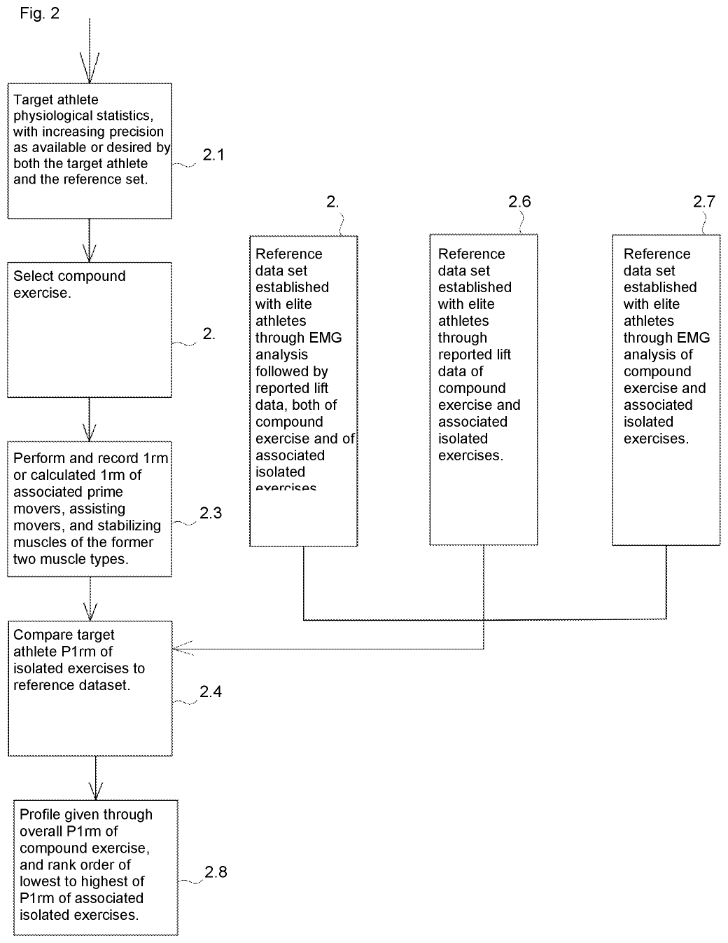

FIG. 2 is a process flowchart showing a method for strength training which calculates the weakest muscles involved and provides a biomechanics analysis, according to the second embodiment of this invention. The difference between the biomechanics analyzing methods of the second and first embodiment resides in the method of providing an analysis with the use of stabilizing muscles.

In this embodiment the P1rm is calculated for each of the prime, assisting, and stabilizing muscles involved in the selected complex exercise (2.3—FIG. 2). Further in analysis (2.8—FIG. 2) the lowest P1rm for the target athlete is determined from the weakest muscle in this group, as well as the rank order of the P1rm of each muscle assessed in isolation for the selected compound movement. Allowing greater precision for more experienced or committed athletes during assessment.

### Third Embodiment

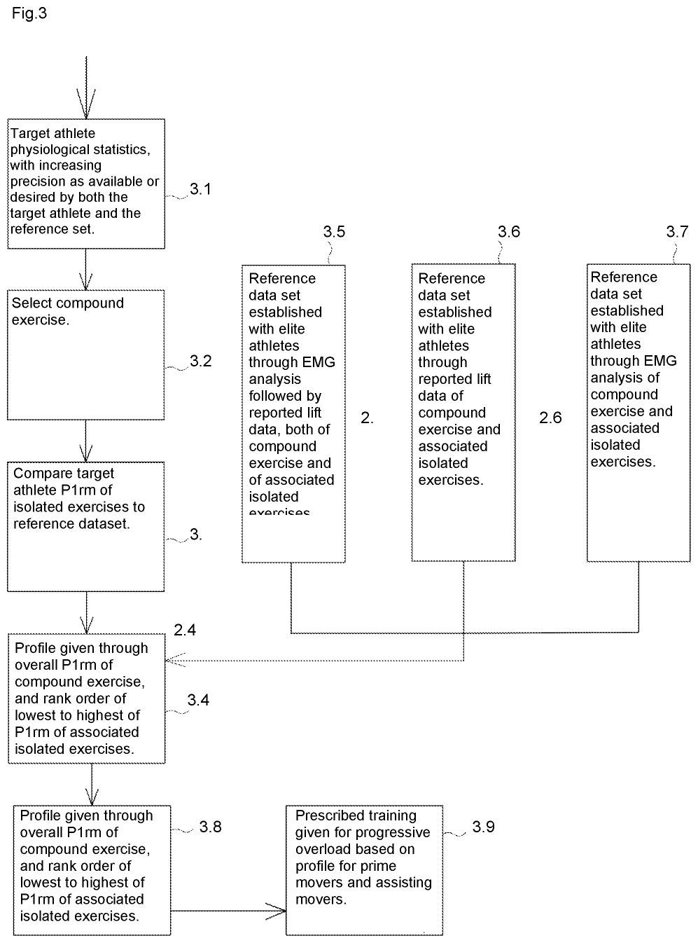

FIG. 3 is a process flowchart showing a method for strength training which calculates the weakest muscles involved and provides a biomechanics analysis, according to the third embodiment of this invention. Furthering on the first embodiment, and as is consistent with the structure of most athletic strength training programs that use progressive overload training, a general assessment can be made to prescribe exercises in terms of core compound movements for use in a progressive overload program. Most commonly the three powerlifting movements of the squat, bench press, and deadlift are used in general strength training for full body strength development. Alternatively this same assessment can be applied to the less frequent olympic movements used similarly in general strength training with progressive overload of power clean, front squat, squat clean, push jerk, power snatch, and squat snatch.

Here, steps one through three remain the same as the first embodiment (3.1, 3.2, 3.3—FIG. 3), however with the given analysis of the fourth step a prescription is made. Using the collective P1rm numbers established by comparing and then selecting the lowest P1rm from a given isolated muscle group, a max weight in a personal training or athletic training regimen as representative of the relatively weakest muscle is given. Typically a progressive overload (increase in weight by low increments) regimen is used for compound movements based on a 1rm assessed as a lift to failure through increments, here the lowest P1rm would replace that assessed 1rm with greater precision by both allowing less fatigue in assessing the 1rm without the use of increments and accounting for the max weight of the lowest muscle as opposed to the collect strength of the muscles while some are over activated relative to others. Then in a rank order the P1rm for each isolated muscle would be categorized from lowest P1rm to highest for prescribing further exercise, from the use of the lowest P1rm first in a typical progressive overload program, for the target athlete to focus their further exercise to the most needed muscles. Stabilizing muscles are properly targeted in the core movement of the progressive overload program given the proper biomechanical ideals of the prime and assisting muscles. Allowing development of biomechanical ideals through training and raising the minimum P1rm faster therefore allowing a faster development of maximum weight used in prescribed training. Thus this prescription is used by the athlete in order of the lowest P1rm for a given compound movement, followed by the lowest recorded P1rm of the prime and assisting muscles in their isolated assessment to the desired effort level of the athlete. For example an assessment of a bench press may give a lowest P1rm of value x followed by a rank of most needed muscles to exercise in order (e.g. triceps, shoulder, pectorals, biceps), and the target athlete may only chose the first one or two of the accuracies after performing the lowest P1rm in the progressive overload program.

### Fourth Embodiment

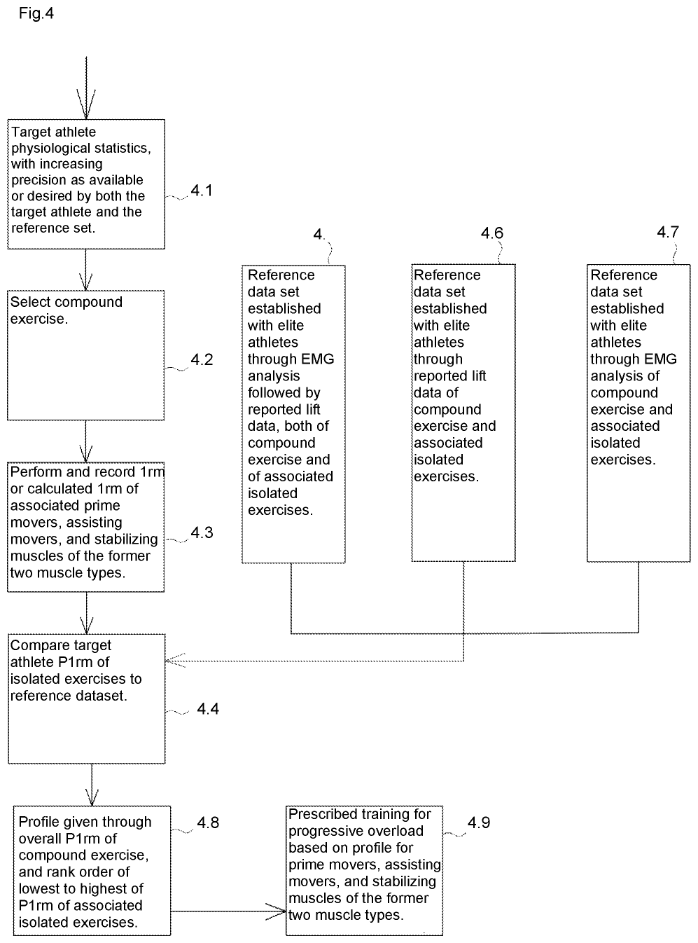

FIG. 4 is a process flowchart showing a method for strength training which calculates the weakest muscles involved and provides a biomechanics analysis, according to the fourth embodiment of this invention. The difference between the biomechanics analyzing methods of the fourth and third embodiment resides in the method of providing an analysis with use of the stabilizing muscles during assessment and prescription of exercise.

In the fourth embodiment, furthering on the third embodiment, in the second step the P1rm assessed for each exercise includes stabilizing muscles (4.3—FIG. 4), as does both lowest P1rm determined through this assessment and rank order of the P1rm of each muscle assessed in isolation for the selected compound movement (4.9—FIG. 4). In the prescription of exercises this allows greater precision for more experienced or committed athletes during assessment.

### Fifth Embodiment

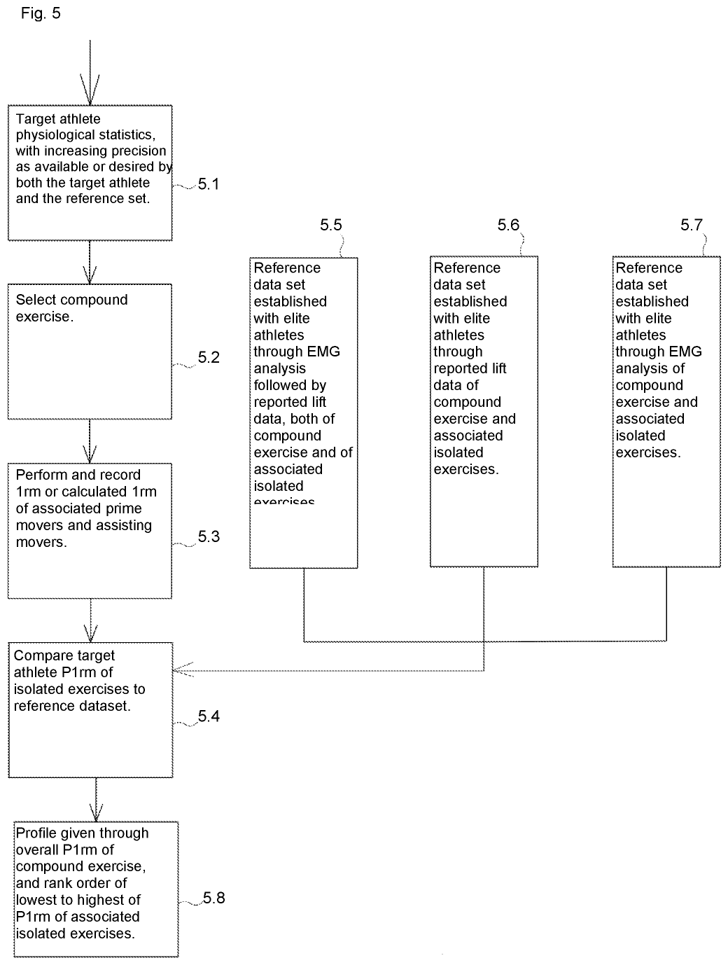

FIG. 5 1 is a process flowchart for this embodiment of the invention; showing a method for a biomechanics analysis for strength training, by establishing both a reference muscular balance profile and further a target athlete profile for comparison, of a selected compound exercise without a total weight lifted. In this case as the P1rm calculation is a measure of relative contribution of the muscles of a compound movement, as determined by EMG activation during the movement, any general athletic movement or movement specific to a sport (e.g. pitching a baseball) may be assessed through the P1rm method. This analysis is done through providing both a rank order and ideal balance ratio of each involved muscle from a reference dataset, and an assessed target athlete for comparison, where (C) in the P1rm formula is represented by the highest weight of any associated muscle's weight lifted in isolation. Due to the nature of assessment it is comprehensive in both accounting for individual physiological factors, and the exercises used during can be calculated through a 1rm formula.

In this embodiment one of three approaches to establishing reference lifts may be used (5.5, 5.6, 5.7—FIG. 5). In all approaches establishing a reference set for a compound movement through the P1rm formula there are two types of movements, compound movements with a total weight lifted (including bodyweight) and compound movements with a force output that is without a max weight lifted (e.g. throwing a pitch). In the first type of movement (C) is represented by the 1rm or calculated 1rm of the actual weight lifted. In the second, as is used in this embodiment, (C) is defined as the highest weight in the reference lifts for all uses, as the overall P1rm is only a rank of the required lifts in determining the biomechanical ideal. For each reference set a desired level of precision could be selected based on the selected, although generally only sex and body weight are necessary, as these allow high precision through data aggregation. Further considerations that can be made are; height, muscle mass, fat mass, visceral fat, fat free mass index, age, bone mass, skeletal muscle mass, maximum rate of oxygen consumption measured during incremental exercise or VO2 max, epigenetic clock, basal metabolic rate, glycemic index, and specific segment weight (limb, torso, etc. . . . ).

The first approach to establishing a reference set for a compound movement through the P1rm formula is an EMG assessment of electrical activation as determined by mean amplitude of the selected compound exercise. This determines the activated muscles during the movement. Then a reference athlete is assessed through working the involved muscles to failure through associated compound exercises in the first type of movement, and through isolated exercises for both types of movements. The 1rm is recorded directly or calculated through a formula. This establishes the strength for each muscle involved in the movement. From here a ratio, (C/S) in the P1rm formula, represents the biomechanical balance of the contributing muscles in isolation to the overall movement, thus a biomechanical ideal. By assessing a small set of reference athletes in the case of EMG assessment to establish an activation reference profile, followed by a larger number of reference athletes through 1rm and calculated 1rm based on the prior activation reference profile a database can be established for a biomechanics reference profile of a given movement.

A second approach, reported lift data, is only the use of reference athletes with a 1rm or calculated 1rm, for which a comparison of a reference athlete's respective total weight lifted and isolated exercises is made in the first type of movement, and with only isolated exercises in the second type of movement. This is limiting in the cases of movements that are not well established in known kinesiology of the muscles comprising the complex movement, unlike the well known core powerlifting movements (e.g. the deadlift's posterior chain muscles are well known).

A third approach is through EMG activation. A reference athlete's respective compound and associated isolated movements are compared with any weight lifted for an associated isolation exercise. By recording the mean electrical amplitude of muscular contraction through electromyography with any weight, and then recording mean amplitude of peak contraction of the associated muscles of the compound exercise in isolation, a comparison of the two amplitudes and the weight in a ratio will predict the total weight that could be lifted by the muscle in isolation. This predicted weight can then be used to establish a reference data set through the P1rm formula.

The P1rm is then calculated for each of the prime and assisting muscles involved in the selected complex exercise, the lowest P1rm for the target athlete is determined from the weakest muscle in this group. In this case as there is no weight loaded for a strength training movement only the rank order of P1rm from lowest to highest is used. This allows development of the muscles associated with the compound or athletic movement to be developed with the proper biomechanics.

The first step in analysis is to have a target athlete's physiological assessment, with increasing precision as desired by and available by both, target athlete and the given reference set. After entering the chosen physiological statistics (5.1—FIG. 5), a matching reference set is chosen. For example if body weight and sex were entered, a reference set based on percentile weight used in a specific exercise at different weight classes and sex (e.g. by 10 lbs for sex), or at a specific bodyweight, is selected for a the balance profile of the selected compound exercise (5.2—FIG. 5).

The second step (5.3—FIG. 5) is recording the target athlete's P1rm for the compound movement through each muscle associated in isolated exercise. P1rm is a novel prediction of a target athlete's 1rm for a given compound exercise, which is calculated without the target athlete actually completing the compound movement. The P1rm calculation can be based on the calculated 1rm, as determined by rep calculator formulas (e.g. Epley formula, Bryzycki formula, or estimation through heart rate), of the various muscles involved in the compound exercise assessed through isolation exercises which here targets the agonist or prime muscles, and synergist or assisting muscle. This isolation allows for a relative level of strength to be determined for each involved muscle with regards to the compound movement. These individual exercises are completed at this step and recorded.

The third step is to take the previous isolated exercises recorded as the P1rm for the compound movement selected, which is done by comparing each individual muscle exercised in isolation to the reference lift dataset, giving an analysis by rank order (5.4—FIG. 5). Again the general formula for Predicted one rep max (P1rm): P1rm=(C/S)*(w), where (C) is the 1rm for a reference athlete or reference athletes for the complex exercise or where (C) is the 1rm or calculated 1rm for a reference athlete or reference athletes for the highest muscle assessed in isolation, (S) is the 1rm for a reference athlete or reference athletes for an isolation exercise associated with the compound exercise, therefore (C/S) is the biomechanical ideal defined by the ratio, and (w) is the calculated or measured 1rm of the isolation exercise. As there is no weight lifted for this method of the formula (C) is defined as the highest weight in the reference lifts for all of the exercises used by the reference athletes, as the use of the P1rm is a rank of the required lifts in determining the biomechanical ideal.

Finally, in the fourth step, an analysis is made (5.8—FIG. 5). by first using the collective P1rm numbers established by comparing and then ranking the lowest P1rm to highest P1rm, of the isolated muscle group of the selected compound exercise. The utility of this embodiment is specific to a single movement on its own, thus prescription of exercises will likely be included into a larger program as one aspect of that program targeted for a specific sport's movement or a therapeutic target.

Thus this prescription is used by the athlete in order of the overall P1rm, followed by the lowest recorded P1rm of the prime and assisting muscles in their isolated assessment to the desired effort level of the athlete. For example a target athlete, like a pitcher assessed, may give a rank of most needed muscles to exercise in order (e.g. posterior deltoid, latissimus dorsi, forearm).

While the fifth embodiment of the invention has been illustrated and described, it will be appreciated that implementation with other methods of health assessment or athletic training would not alter the spirit or scope of the invention. For example implementation into a larger health application or exercise program that uses muscular assessment as part of a larger program for either exercise or health tracking or prescription. Additionally one of ordinary skill will recognize that the step by step process of this embodiment is a calculation that can be readily implemented by a computer.

### Sixth Embodiment

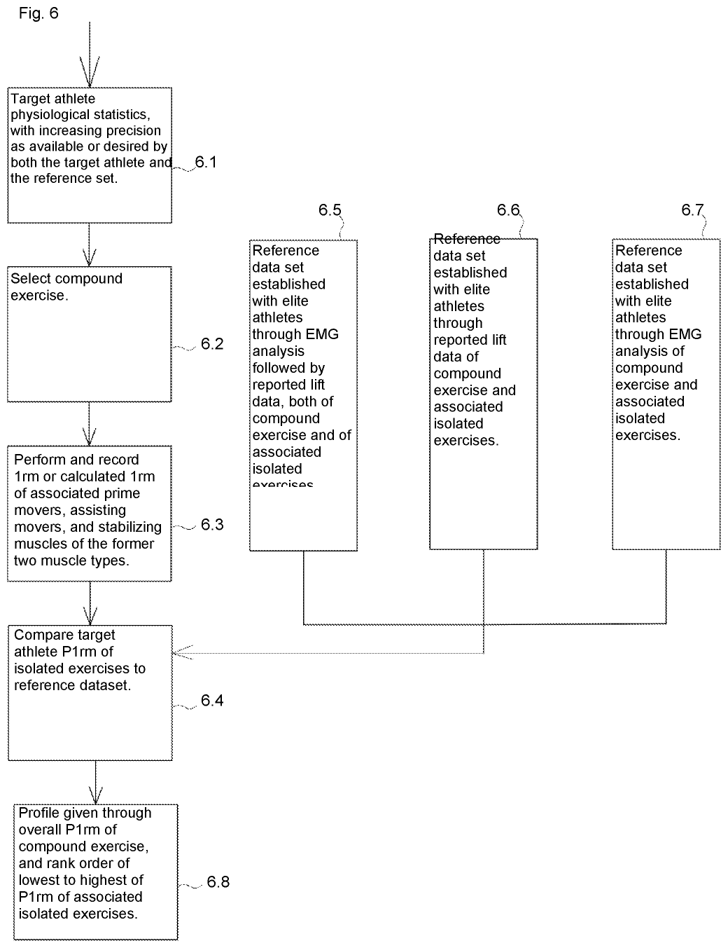

FIG. 6 is a process flowchart showing a method for strength training which calculates the weakest muscles involved and provides a biomechanics analysis, according to the sixth embodiment of this invention. The difference between the biomechanics analyzing methods of the sixth and fifth embodiment resides in the method of providing an analysis with use of the stabilizing muscles during assessment and prescription of exercise.

Furthering on the third embodiment, in the second step the P1rm assessed for each exercise includes stabilizing muscles (6.3—FIG. 6), as does the rank order of the P1rm of each muscle assessed in isolation for the selected compound movement (6.8—FIG. 6), allowing greater precision for more experienced or committed athletes during assessment.

### INDUSTRIAL APPLICABILITY

Athletic training
Personal fitness training
Physical therapy

### CITATION LIST

Croisier, S. L., Ganteaurne, S., Binet, J., Genty, M., & Ferret, J. M. (2008). Strength imbalances and prevention of hamstring injury in professional soccer players: a prospective study. The American journal of sports medicine, 36(8), 1469-1475. https://doi.org/10.1177/0363546508316764
Poliquin, C. (1997). Poliquin principles: successful methods for strength and mass development
Nation, C. T., T. (2015, February 26). Know Your Ratios, Destroy Weaknesses. T NATION. https://www.t-nation.com/training/know-your-ratios-destroy-weaknesses/Richens,
B., & Cleather, D. (2014). The relationship between the number of repetitions performed at given intensities is different in endurance and strength trained athletes Biology of Sport, 31(2), 157-161.

1. A biomechanics process for a compound exercise compromising the steps of: selecting a compound exercise with a total weight lifted; using a reference athlete or athletes to record a one rep max or calculated one rep max of the compound exercise; during which associated prime and assisting muscles of the compound exercise are determined through, either mean amplitude of electrical activity of the muscles determined through electromyography, or exercise science literature of kinesiology analysis, or both any weight lifted and the mean electrical amplitude of muscular contraction through electromyography then recording mean amplitude of peak contraction of the associated prime and assisting muscles before comparing the two amplitudes and weight in a ratio to determine the possible peak weight; further wherein reference athletes record a one rep max or calculated one rep max of the isolated exercises, of the associated prime and assisting muscles; so that a muscular balance profile of the compound exercise is created through a rank order of the exercises assessed through the formula [P1rm=(C/S)*(w)]; where (C) is the 1rm or calculated 1rm for a reference athlete or reference athletes for the complex exercise, (S) is the 1rm or calculated 1rm for a reference athlete or reference athletes for the associated prime or assisting muscle of the compound exercise, (C/S) is the biomechanical ideal defined by the ratio, and (w) is the 1rm or calculated 1rm of an isolated exercise of the associated prime or assisting muscle of the compound exercise completed by the reference athlete.

2. The method of claim 1 further consisting of: exercise by a target athlete; recording the one rep max or calculated one rep max of the associated prime and assisting muscles of a compound exercise selected in claim 1; wherein prime and assisting muscles are used for analysis by comparison to the muscular balance profile of claim 1; through the formula [P1rm=(C/S)*(w)], where (C) is the 1rm or calculated 1rm for a reference athlete or reference athletes for the complex exercise, (S) is the 1rm or calculated 1rm of the target for the associated prime or assisting muscle of the compound exercise, (C/S) is the biomechanical ideal defined by the ratio, and (w) is the 1rm or calculated 1rm of an isolated exercise of the associated prime or assisting muscle; allowing a muscular balance profile of prime and assisting muscles of the target athlete, by each muscle of the compound movement through, its isolated assessment inputted into the P1rm formula, providing both rank order and ratio compared to the reference ideal.

3. A biomechanics process for a compound exercise compromising the steps of: selecting a compound exercise with a total weight lifted; using a reference athlete or athletes to record a one rep max or calculated one rep max of the compound exercise; during which associated prime, assisting, and stabilizing muscles of the compound exercise are determined through, either mean amplitude of electrical activity of the muscles determined through electromyography during, or exercise literature of kinesiology analysis, or both any weight lifted and the mean electrical amplitude of muscular contraction through electromyography then recording mean amplitude of peak contraction of the associated prime, assisting, and stabilizing muscles before comparing the two amplitudes and weight in a ratio to determine the possible peak weight; further wherein reference athletes record a one rep max or calculated one rep max of the isolated exercises of the associated prime, assisting, and stabilizing muscles; so that a muscular balance profile of the compound exercise is created through a rank order of the exercises assessed through the formula [P1rm=(C/S)*(w)]; where (C) is the 1rm or calculated 1rm for a reference athlete or reference athletes for the complex exercise, (S) is the 1rm or calculated 1rm for a reference athlete or reference athletes for the associated prime, assisting, and stabilizing muscles of the compound exercise, (C/S) is the biomechanical ideal defined by the ratio, and (w) is the 1rm or calculated 1rm of an isolated exercise of the associated prime, assisting, and stabilizing muscles of the compound exercise completed by the reference athlete.

4. The method of claim 3 further consisting of: exercise by a target athlete; recording the one rep max or calculated one rep max of the associated prime, assisting, and stabilizing muscles of a compound exercise assessed as in claim 3; wherein prime, assisting, and stabilizing muscles are used for analysis by comparison to the muscular balance profile of claim 3; through the formula [P1rm=(C/S)*(w)], where (C) is the 1rm or calculated 1rm for a reference athlete or reference athletes for the complex exercise, (S) is the 1rm or calculated 1rm of the target athlete for the associated prime, assisting, and stabilizing muscles of the compound exercise, (C/S) is the biomechanical ideal defined by the ratio, and (w) is the 1rm or calculated 1rm of an isolated exercise of the associated prime, assisting, and stabilizing muscles; allowing a muscular balance profile of prime and assisting muscles of the target athlete, by each muscle of the compound movement through, its isolated assessment inputted into the P1rm formula, providing both rank order and ratio compared to the reference ideal.

5. The method of claim 2 or 4 further consisting of: use by the target athlete, of the lowest P1rm value of any associated exercise of a muscle in isolation, in place of a one rep max until failure in determining the max weight for a progressive overload strength training program of the selected compound exercise; as well as the rank order of the P1rm of each muscle in isolation, being used to program the accessory work in a progressive overload strength training program, by prioritizing the relatively weakest muscles of the selected compound exercise.

6. A biomechanics process for a compound exercise compromising the steps of: selecting a compound exercise without a total weight lifted; using a reference athlete or athletes to perform the movement; during which associated prime and assisting muscles of the compound exercise are determined through, either mean amplitude of electrical activity of the muscles determined through electromyography, or exercise literature of kinesiology analysis, or both any weight lifted and the mean electrical amplitude of muscular contraction through electromyography then recording mean amplitude of peak contraction of the associated prime and assisting muscles before comparing the two amplitudes and weight in a ratio to determine the possible peak weight; further wherein reference athletes record a one rep max or calculated one rep max of the isolated exercises of the associated prime and assisting muscles; so that a muscular balance profile of the compound exercise is created through a rank order of the exercises assessed through the formula [P1rm=(C/S)*(w)]; where (C) is the 1rm or calculated 1rm for a reference athlete or reference athletes for the highest muscle assessed in isolation, (S) is the 1rm or calculated 1rm for a reference athlete or reference athletes for the associated prime or assisting muscles of the compound exercise, (C/S) is the biomechanical ideal defined by the ratio, and (w) is the 1rm or calculated 1rm of an isolated exercise of the associated prime or assisting muscle of the compound exercise completed by the reference athlete.

7. The method of claim 6 further consisting of: exercise by a target athlete; recording the one rep max or calculated one rep max of the associated prime and assisting muscles of a compound exercise selected in claim 6; wherein prime and assisting muscles are used for analysis by comparison to the muscular balance profile of claim 6; through the formula [P1rm=(C/S)*(w)], (C) is the 1rm or calculated 1rm for a reference athlete or reference athletes for the highest muscle assessed in isolation, (S) is the 1rm or calculated 1rm of the target athlete for the associated prime or assisting muscles of the compound exercise, (C/S) is the biomechanical ideal defined by the ratio, and (w) is the 1rm or calculated 1rm of an isolated exercise of the associated prime or assisting muscle; allowing a muscular balance profile of prime and assisting muscles of the target athlete, by each muscle of the compound movement through, its isolated assessment inputted into the P1rm formula, providing both rank order and ratio compared to the reference ideal.

8. A biomechanics process for a compound exercise compromising the steps of: selecting a compound exercise without a total weight lifted; using a reference athlete or athletes to perform the movement; during which associated prime, assisting, and stabilizing muscles of the compound exercise are determined through, either mean amplitude of electrical activity of the muscles determined through electromyography, or exercise literature of kinesiology analysis, or both any weight lifted and the mean electrical amplitude of muscular contraction through electromyography then recording mean amplitude of peak contraction of the associated prime, assisting, and stabilizing muscles before comparing the two amplitudes and weight in a ratio to determine the possible peak weight; further wherein reference athletes record a one rep max or calculated one rep max of the isolated exercises of the associated prime, assisting, and stabilizing muscles; so that a muscular balance profile of the compound exercise is created through a rank order of the exercises assessed through the formula [P1rm=(C/S)*(w)]; where (C) is the 1rm or calculated 1rm for a reference athlete or reference athletes for the highest muscle assessed in isolation, (S) is the 1rm or calculated 1rm for a reference athlete or reference athletes for the associated prime, assisting, and stabilizing muscles of the compound exercise, (C/S) is the biomechanical ideal defined by the ratio, and (w) is the 1rm or calculated 1rm of an isolated exercise of the associated prime, assisting, and stabilizing muscles of the compound exercise completed by the reference athlete.

9. The method of claim 6 further consisting of: exercise by a target athlete; recording the one rep max or calculated one rep max of the associated prime, assisting, and stabilizing muscles of a compound exercise selected in claim 6; wherein prime, assisting, and stabilizing muscles are used for analysis by comparison to the muscular balance profile of claim 6; through the formula [P1rm=(C/S)*(w)], where (C) is the 1rm or calculated 1rm for a reference athlete or reference athletes for the highest muscle assessed in isolation, (S) is the 1rm or calculated 1rm of the target athlete for the associated prime, assisting, and stabilizing muscles of the compound exercise, (C/S) is the biomechanical ideal defined by the ratio, and (w) is the 1rm or calculated 1rm of an isolated exercise of the associated prime, assisting, and stabilizing muscles; allowing a muscular balance profile of prime, assisting, and stabilizing muscles of the target athlete, by each muscle of the compound movement through, its isolated assessment inputted into the P1rm formula, providing both rank order and ratio compared to the reference ideal.
